(19) United States
(12) Patent Application Publication
(10) Pub. No.: US 2009/0136390 A1
(43) Pub. Date: May 28, 2009

(54) INCANDESCENT AROMA LAMP

(76) Inventor: CHIN-SHENG YANG, TAINAN CITY (TW)

Correspondence Address: LEONG C LEI, PMB # 1008, 1867 YGNACIO VALLEY ROAD, WALNUT CREEK, CA, 94598, US

(21) Appl. No.: 12/241,080

(22) Filed: Sep. 30, 2008

(30) Foreign Application Priority Data
Nov. 26, 2007 (TW) 096219936

(51) Int. Cl. A61L 9/03

(52) U.S. Cl. 422/125

(57) ABSTRACT

The incandescent aroma lamp contains a base member having an opening and a plug. An incandescent light bulb is housed inside the base member. An indented support frame covers the top opening of the base member and supports an aroma producing element in the indentation. A cap is detachably positioned on top of the base member and covers the aroma producing element. When the plug is connected to an electrical socket, the incandescent light bulb is turned on and drives air by thermal convection to flow upward against the aroma producing element. The aroma producing element vaporizes and the aroma is spread outside the cap.

### TECHNICAL FIELD OF THE INVENTION

The present invention generally relates to aroma or fragrance producing devices, and more particularly to an aroma producing device having an incandescent light bulb to provide illumination as well.

### DESCRIPTION OF THE PRIOR ART

There are various commercially available devices for removing unpleasant odors from the environment, from pets, from dirty clothes, etc. For example, there are incense burning devices, deodorant spraying devices, ethereal oil burning devices, to name just a few. Recently, the ethereal oil burning devices are especially popular as aroma therapy receives wide acceptance as an effective healing and relaxing means.

On the other hand, there are also night lamps that are usually plugged into a wall outlet and use a light bulb of small wattage or a light emitting diode for illumination. These night lamps usually have visually appealing bodies or caps, so that they have decorative function as well. If a night lamp is integrated with an ethereal oil burning device, the product would have an enhanced market value.

### SUMMARY OF THE INVENTION

Therefore, a novel incandescent aroma lamp is provided herein which simultaneously provides illumination and aroma. The incandescent aroma lamp contains a hollow base member having a top opening and a plug. An incandescent light bulb is housed inside the base member. An indented support frame covers the top opening of the base member and supports an aroma producing element in its indentation. A cap is detachably joined to the base member to cover the top opening, the aroma producing element, and the support frame.

When the plug is connected to an electrical outlet, the incandescent light bulb is turned on by the electricity drawn through the plug. The incandescent light bulb drives air by thermal convection to flow upward against the aroma producing element. The aroma producing element vaporizes and the aroma is spread outside the cap via a top opening of the cap.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective diagram showing the various components of an aroma lamp according to an embodiment of the present invention.

FIG. 2 is a perspective diagram showing the aroma lamp of FIG. 1 after its assembly.

FIG. 3 is a sectional diagram showing the aroma lamp of FIG. 1.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

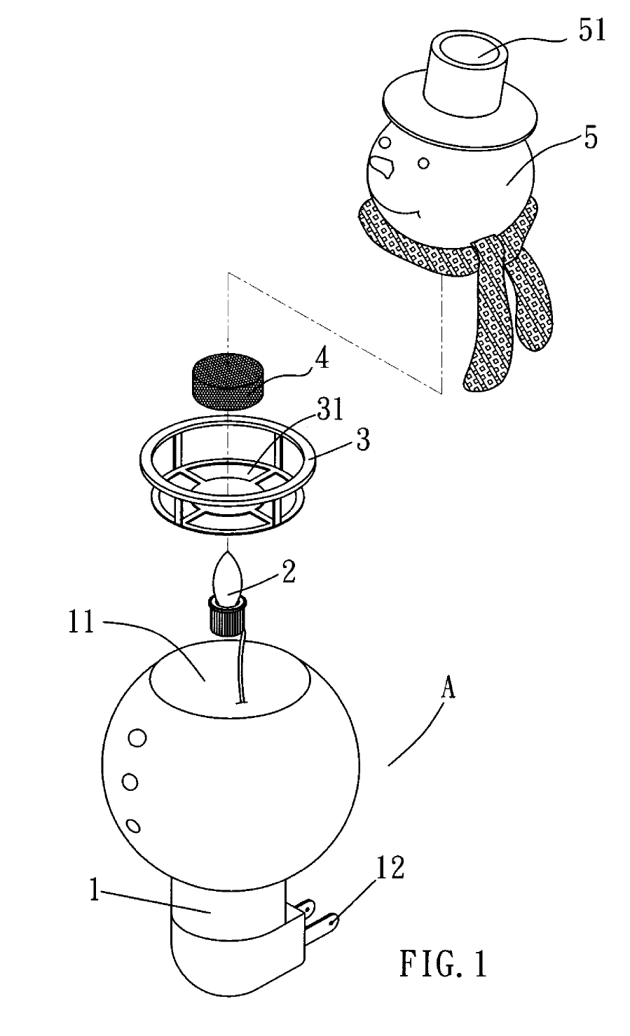

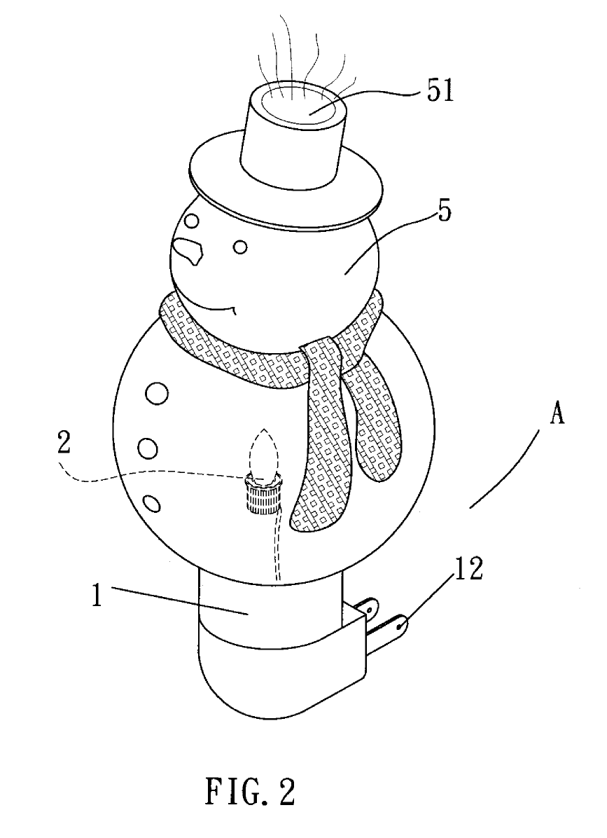

As shown in FIGS. 1 and 2, an incandescent aroma lamp A according to an embodiment of the present invention mainly contains a hollow base member 1 having a top opening 11 and a plug 12. By plugging the plug 12 to an electrical socket such as a wall socket, the incandescent aroma lamp A draws electricity from the socket to power its components.

An incandescent light bulb 2 is housed inside the base member 1. When the incandescent light bulb 2 is turned on by the electricity drawn through the plug 12, it will produce light for illumination and thermally convected air flow.

The incandescent aroma lamp A further contains a support frame 3 which covers the top opening 11 of the base member 1. The support frame 3 has a downward indentation and, surrounding the indentation, the support frame 3 has a number of openings 31 allowing air to flow through.

An aroma producing element 4 is supported by the support frame 3 inside the indentation and is therefore positioned right above the incandescent light bulb 2.

A cap 5 is positioned on top of the base member 1, covering the top opening 11, the support frame 3, and the aroma producing element 4. There is an opening 51 on a top side of the cap 5 to allow air flow.

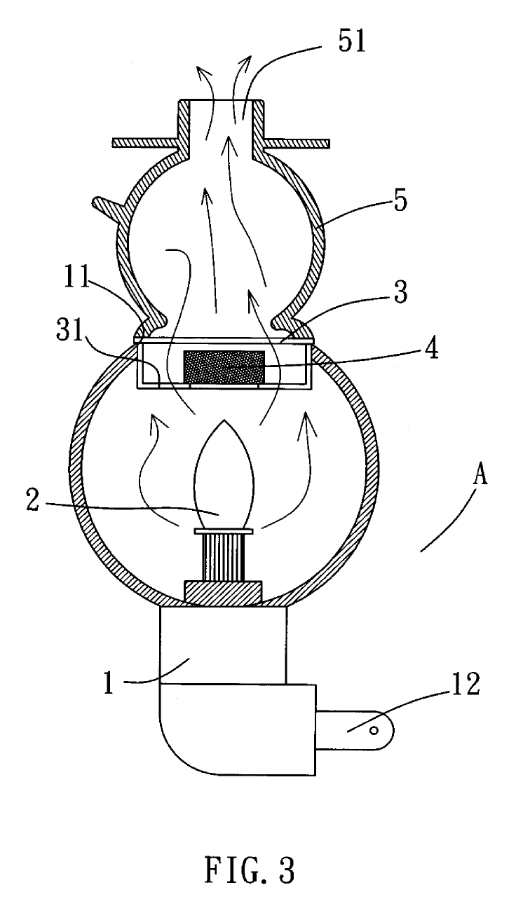

As also shown in FIG. 3, when the plug 12 is plugged to a wall socket, the incandescent light bulb 2 is turned on by the electricity drawn through the plug 12. In addition to the illumination provided by the incandescent light bulb 2, the heat produced by the incandescent light bulb 2 drives air to flow upward against the aroma producing element 4 via the openings 31 of the support frame 3. The aroma producing element 4 vaporizes under the heat and the aroma is spread along the convected air flow outside the cap 5 via its top opening 51. The aroma producing element 4 could be a sponge soaked with ethereal oil or a soap made of solidified ethereal oil.

Please note that the cap 5 could have various visually appealing shapes. The cap 5 is detachably joined to the base member 1 and could be removed easily so that the support frame 3 and the base member 1 could be used by themselves. The incandescent light bulb 2 is connected to the plug 12 via a simple circuit. The details to the circuit should be straightforward to people of related arts and are therefore omitted here.

As described above, the aroma device of the present invention simultaneously provides illumination and aroma. It uses a vaporizable aroma producing element as the source of aroma and utilizes the heat and convected air flow from the incandescent light bulb to facilitate its vaporization and the delivery of aroma.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An incandescent aroma lamp, comprising:
a hollow base member having a top opening and a plug;
an incandescent light bulb housed inside said base member and electrically connected to said plug;
a support frame having a downward indentation joined to said base member to cover said top opening of said base member, said support frame having a plurality of openings surrounding said indentation to allow air to flow through;
an aroma producing element positioned inside said indentation of said support frame; and
a cap positioned on top of said base member to cover said aroma producing element, said support frame, and said top opening of said base member, said cap having a top opening;
wherein said incandescent light bulb is turned on by electricity drawn through said plug by plugging said plug to an electrical outlet; said incandescent light bulb drives air to flow upward by convection against said aroma producing element via said openings of said support frame; said aroma producing element vaporizes and aroma is spread outside said cap via said top opening of said cap.

2. The incandescent aroma lamp according to claim 1, wherein said aroma producing element is a sponge soaked with ethereal oil.

3. The incandescent aroma lamp according to claim 1, wherein said aroma producing element is a soap made of ethereal oil.
